(19) United States
(12) Patent Application Publication
(10) Pub. No.: US 2012/0289452 A1
(43) Pub. Date: Nov. 15, 2012

(54) COMPOSITIONS AND METHODS FOR TREATING AND PREVENTING NECROSIS

(75) Inventors: Ilana NATHAN, Omer (IL); Alexandra Lichtenstein, Beer-Sheva (IL)

(73) Assignee: Ilana Nathan, Omer (IL)

(21) Appl. No.: 13/164,870

(22) Filed: Jun. 21, 2011

Related U.S. Application Data
(63) Continuation of application No. 10/509,405, filed on Sep. 24, 2004, now abandoned.

(30) Foreign Application Priority Data
Mar. 26, 2002 (IL) 148924

(51) Int. Cl. A61K 38/14; A61P 35/02; A61P 11/00; A61P 3/10; A61P 31/06; A61P 9/00; A61P 17/06; A61P 9/10; A61P 1/18; A61P 31/14; A61P 7/00; A61P 31/04; A61P 17/02; A61P 1/00; A61P 29/00; A61P 19/02; A61P 3/00; A61P 31/12; A61P 25/32; A61P 25/28

(52) U.S. Cl. 514/1.4; 514/20.4; 514/17.7; 514/19.6; 514/1.5; 514/6.9; 514/2.4; 514/18.7; 514/16.4; 514/4.3; 514/13.5; 514/18.6; 514/1.8; 514/16.6; 514/17.9; 514/16.7; 514/3.7

(57) ABSTRACT

A method for treating and/or preventing cell necrosis and diseases associated therewith, comprising the inhibition of one or more elastase enzymes within said cells.

### FIELD OF THE INVENTION

The present invention relates to methods and compositions for treating and preventing cell necrosis. More specifically, the methods and compositions of the present invention prevent or treat necrosis by means of inhibiting the activity of intracellular elastase acting in the cells undergoing necrosis.

### BACKGROUND OF THE INVENTION

Elastase is a serine protease that catalyses the degradation of proteins, including elastin, a major structural protein of mammalian connective tissue. The art has suggested that the inhibition of elastase may be effective in the treatment of various conditions and diseases.

For example, U.S. Pat. No. 4,683,241 indicates that elastase is believed to play an important role in the etiology of inflammatory connective tissue diseases. This patent discloses a class of phenolic esters exhibiting elastase inhibitory action.

U.S. Pat. No. 5,216,022 discloses the use of aromatic esters of phenylenedialkanoates as inhibitors of human neutrophil elastase (also known as leukocyte elastase), for treating numerous neutrophil elastase-mediated conditions.

U.S. Pat. No. 6,159,938 indicates that the inhibition of endogenous vascular elastase may be effective in treating pulmonary vascular disease and other related conditions.

Necrosis is the relatively uncontrolled process of cell death following perturbation to the cellular environment, resulting in cell rupture. Necrosis may be treated by the use of high pressure oxygen.

### SUMMARY OF THE INVENTION

The inventors have unexpectedly found that intracellular elastase is involved in necrotic cell death, and that the inhibition of said enzyme within the affected cells may serve as an effective tool for treating and/or preventing cell necrosis and diseases associated therewith.

The present invention provides a method for treating and preventing necrosis of cells and diseases associated therewith, comprising inhibiting the enzymatic activity of one or more elastase enzymes within said cells.

In one aspect, the above mentioned method comprises administering to a subject a therapeutically effective amount of one or more elastase inhibiting agents, wherein said agents inhibit the enzymatic activity of intracellular elastase in the cells to be treated.

The present invention also encompasses a method for inhibiting and preventing cell necrosis in vitro, comprising causing an effective amount of one or more elastase inhibitors to enter the cells to be treated.

The inventors have also surprisingly found the inhibition of elastase within the affected cells may shift cell necrosis, at least partially, into apoptotic cell death. Thus, in a preferred embodiment, the invention provides a method for treating and preventing cell necrosis and diseases associated therewith, comprising:

inhibiting the enzymatic activity of elastase within said cells; and
inhibiting apoptotic cell death.

The present invention is also directed to pharmaceutical compositions for the treatment and/or prevention of cell necrosis and diseases associated therewith, wherein said compositions comprise therapeutically effective amounts of one or more agents that inhibit the enzymatic activity of one or more elastase enzymes in the cells to be treated. Thus, the abovementioned pharmaceutical compositions comprise one or more elastase inhibitors that are capable of entering the cells to be treated, in combination with one or more suitable pharmaceutically-acceptable excipients.

According to one preferred embodiment of the invention, the abovementioned pharmaceutical compositions further comprise one or more inhibitors of apoptosis.

In a further aspect of the present invention is provided the use of one or more elastase inhibitors in the preparation of a medicament for treating and/or preventing necrosis of cells and diseases associated therewith, wherein said elastase inhibitors are capable of entering said cells.

In a preferred embodiment, the invention is also directed to the use of one or more elastase inhibitors together with one or more inhibitors of apoptosis in the preparation of a medicament for treating and/or preventing necrosis of cells and diseases associated therewith, wherein said elastase inhibitors are capable of entering said cells.

The inhibitors of elastase activity used according to the invention for treating and preventing cell necrosis, and diseases associated therewith, are all capable of entering into the target cells, such that said inhibitors exert their inhibitory actions within said cells.

Preferably, necrosis may be treated or prevented according to the present invention in cells selected from the group consisting of neuronal cells, purkinje cell, hypocampal pyramidal cells, glial cells, cells of hematopoetic origin (such as lymphocytes and macrophages), hepatocytes, thymocytes, fibroblast, myocardial cells, epithelial cells, bronchial epithelial cells, glomeruli, lung epithelial cells, keratinocytes, gastrointestinal cells, epidermal cells, bone and cartilage cells.

Preferably, the diseases associated with cell necrosis, which may be treated and/or prevented according to the present invention, are selected from the group consisting of neurodegenerative disorders, leukemias, lymphomas, neonatal respiratory distress, asphyxia, incarcerated hernia, diabetes mellitus, tuberculosis, endometriosis, vascular dystrophy, psoriasis, cold injury, iron-load complications, complications of steroid treatment, ischemic heart disease, reperfusion injury, cerebrovascular disease or damage, gangrene, pressure sores, pancreatitis, hepatitis, hemoglobinuria, bacterial sepsis, viral sepsis, burns, hyperthermia, Crohn's disease, celiac disease, compartment syndrome, necrotizing procolitis, cystic fibrosis, rheumatoid arthritis, nephrotoxicity, multiple sclerosis, spiral cord injury, glomerulonephritis, muscular dystrophy, degenerative arthritis, tyrosemia, metabolic inherited disease, mycoplasmal disease, anthrax infection, infection with other bacteria, viral infections, Anderson disease, congenital mitochondrial disease, phenylketonuria, placental infarct, syphilis, aseptic necrosis, avascular necrosis, alcoholism and necrosis associated with administration and/or self-administration with, and/or exposure to, cocaine, drugs (e.g., paracetamol, antibiotics, adriamycin, NSAID, cyclosporine) chemical toxins such as carbon tetrachloride, cyanide, methanol, ethylene glycol and mustard gas, agrochemicals such organophosphats and paraquat, heavy metals (lead, mercury), other warfare organophosphats.

In another embodiment, the composition and methods of the invention may be used for the treatment and/or prevention of aging, by inhibiting the enzymatic activity of one or more elastase enzymes, more particularly the intracellular activity thereof, optionally together with the inhibition of apoptosis and the use of anti-aging agents.

### BRIEF DESCRIPTION OF THE DRAWINGS

FIG. 1 graphically depicts the percentage of necrotic and apopoptic cells observed following treatment with and without oligomycin and anti-Fas.

FIG. 2 is a photographic representation of gelatin substrate gel electrophoresis results for lysates of U-937 cells treated/untreated with oligomycin and/or anti-Fas for 3 hours.

FIG. 3 is a photographic representation of gelatin substrate gel electrophoresis results obtained for lysates of U-937 cells treated/untreated with 0.5 mM KCN for 3 hours.

FIG. 4 is a photographic representation of a gelatin substrate electrophoretic gel, demonstrating that treatment of a cell lysate with KCN caused the appearance of a band of protease activity (lane B). This band disappeared when KCN was administered in the presence of 200 μM elastase inhibitor (lane C).

FIG. 5 presents results demonstrating the effect of elastase inhibitor III on KCN-induced necrosis in PC-12 cells. Panel A diagrammatically depicts the proportion of live, necrotic and apoptotic cells following various treatments. The numerical values for these proportions are given in the accompanying table. Panel B graphically depicts percentage PC-12 cell survival following treatment with KCN in the presence/absence of elastase inhibitor III.

FIG. 6 diagrammatically depicts the proportion of live, necrotic and apoptotic U-937 cells following treatment with KCN in the presence/absence of elastase inhibitor III. The numerical values for these proportions are given in the accompanying table.

FIG. 7 graphically illustrates the effects of elastase inhibitor III (panel B) and elastinal (Panel C) on Fas-induced apoptosis/necrosis in U-397 cells.

FIG. 8 graphically illustrates the percentage of necrotic and apoptotic PC-12 cells detected following treatment with/without oligomycin and/or STS.

FIG. 9 demonstrates the effect of an elastase inhibitor on STS-induced apoptosis in PC-12 cells.

FIG. 10 graphically illustrates the effect of an elastase inhibitor on STS-induced necrosis in PC-12 cells.

FIG. 11 demonstrates the effect of an elastase inhibitor on KCN-induced necrosis in PC-12 cells.

FIG. 12 graphically illustrates the effect of an elastase inhibitor on STS-induced necrosis in U-937 cells.

### DETAILED DESCRIPTION OF PREFERRED EMBODIMENTS

The term “necrosis”, as used herein, encompasses cell necrosis states, as well as intermediates states, exhibiting necrotic and apoptotic characteristics. The term “elastase”, as used herein, refers to one or more forms of said enzyme.

Compounds exhibiting elastase inhibitory profile, which are herein referred to as elastase inhibiting agents, or elastase inhibitors, are known in the art, and are disclosed, for example, by Stein et. al. [Biochemistry 25, p. 5414 (1986)], Powers et al. [Biochim. Biophys. Acta. 485, p. 15 (1977)], U.S. Pat. No. 4,683,241, U.S. Pat. No. 5,216,022, and U.S. Pat. No. 6,159,938. Inhibitors of elastase are also commercially available from, e.g., Sigma-Aldrich or Calbiochem-Novabiochem Corporation.

Elastase inhibitors used according to the present invention are formulated together with one or more pharmaceutically acceptable carriers, which are non-toxic, inert solid, semi-solid or liquid fillers, diluent, encapsulating material or formulation auxiliary of any type. The pharmaceutical compositions can be administered to human and other mammalian subjects in any acceptable route, and preferably orally, parenterally or topically.

Solid dosage forms for oral administration include capsules, tablets, pills, powders and granules. In such solid dosage forms, the active compound is mixed with at least one inert, pharmaceutically acceptable excipient or carrier such as sodium citrate or dicalcium phosphate and/or fillers or extenders such as starches, lactose, sucrose, glucose and mannitol, binders such as carboxymethylcellulose and gelatin, humectants such as glycerol, disintegrating agents such as agar-agar, calcium carbonate and potato starch, absorbents and lubricants. The solid dosage forms can be prepared with coatings and shells according to methods known in the art.

Liquid dosage forms for oral administration include pharmaceutically acceptable solutions, emulsions, suspensions and syrups. In addition to the active compounds, the liquid dosage form may contain inert diluents commonly used in the art such as water or other solvents, solubilizing agents and emulsifiers such as ethyl alcohol, isopropyl alcohol, ethyl carbonate, ethyl acetate, propylene glycol and oils. Besides inert diluents, the oral compositions may also include adjuvants such as wetting agents, emulsifying and suspending agents, sweetening, flavoring and perfuming agents.

Injectable preparations suitable for parenteral administration are provided in the form of pharmaceutically acceptable sterile aqueous or non-aqueous solutions, dispersions, suspensions or emulsions as well as sterile powders for reconstitution into sterile injectable solutions or dispersions prior to use. Examples of suitable aqueous or non-aqueous carriers or vehicles include water, Ringer's solution and isotonic sodium chloride solution. Sterile oils may also be employed as a suitable suspending medium. The injectable formulations can be sterilized, for example, by filtration through a bacterial-retaining filter, or by incorporating sterilizing agents therein.

Dosage forms for topical or transmucosal administration of elastase inhibitors according to the invention may include pastes, creams, lotions, gels, powders, solutions and sprays. In addition to the active ingredient, the pastes creams and gels may contain excipients such as fats, oils, waxes, paraffins, starch, cellulose derivatives, polyethylene glycols, talc, zinc oxide, or mixture thereof. Powders and sprays can contain excipient such as lactose, talcs, silicic acid, aluminum hydroxide, calcium silicates and mixtures thereof.

It should be noted that in addition to the medical or pharmaceutical use of topical and transmucosal compositions containing elastase inhibitors (and optionally, anti-apoptotic agents), the present invention also provides said compositions for use as cosmetic agents.

Other suitable formulations may be prepared by encapsulating the active ingredient in lipid vesicles or in biodegradable polymeric matrices, or by attaching said active ingredient to monoclonal antibodies. Methods to form liposomes are known in the art.

Dosage levels of active ingredients in the pharmaceutical compositions of this invention may be varied so as to obtain an amount of the elastase inhibitor that is effective to achieve the desired therapeutic response for a particular patient (i.e., a therapeutically effective amount). The selected dosage form will depend on the activity of the particular elastase inhibitor, the route of administration, the severity of the condition being treated and other factors associated with the patient being treated. Typical dose regimes are in the range of 0.1-200 mg/kg.

In another aspect, the present invention is directed to the treatment or prevention of cell necrosis by means of inhibiting the enzymatic activity of intracellular elastase(s), and, in addition, inhibiting apoptotic cell death. In a preferred embodiment of this aspect of the invention, the inhibition of apoptotic cell death is accomplished by means of administering to subject a therapeutic effective amount of an anti-apoptotic agent, which is preferably selected from the group consisting of [R]—N-[2-heptyl]-methylpropargylamine (R-2HMP), vitamin E, vitamin D, caspase inhibitors and the hydrophilic bile salt ursodeoxycholic acid. Other methods known in the art for inhibiting apoptosis, for example, by means of regulation of expression of pro- and anti-apoptotic proteins, may also be used according to the present invention. Such methods are described, for example, by Li et al. [Acta. Anaesthesiol Sin, 38(4), p. 207-215 (2000)].

### EXAMPLES

### Experimental Protocol

### 1. Models of Necrosis In Vitro

### Staurosporine and Anti-Fas-Induced Necrosis

Human promonocytic U-937 cells in logarithmic phase were seeded at a concentration of 4×10⁵/ml. Afterwards the cells were washed twice and seeded again in glucose-free RPMI-1640 medium (Beit Haemek, Israel) supplemented with 2 mM pyruvate (Beit Haemek, Israel) and 10% dialyzed FCS (Gibco, BRL) for one hour.

The rat pheochromocytoma PC-12 cell line was propagated in DMEM medium (Gibco, BRL), supplemented with 5% heat-inactivated calf serum, 10% heat-inactivated horse serum, and 2 mM L-glutamine. PC-12 cells in logarithmic phase were seeded at a concentration of 1.2×10⁵/well in 24-well plates (Cellstar). Then the cells were washed twice and maintained in glucose-free RPMI-1640 medium (Beit Haemek, Israel), and supplemented with 2 mM pyruvate and 10% dialyzed FCS for one hour. U-937 and PC-12 cells were incubated with and without 1 μM oligomycin (Sigma) for 45 min, and cells were treated with or without 1.25 μM staurosporine (STS) (Sigma) for an additional seven hours in U-937 cells or five hours in PC-12 cells. Alternatively, cells were treated with or without 100 ng/ml anti-Fas (Upstate biotechnology, USA) for the same time period.

### KCN-Induced Necrosis

U-937 and PC-12 cells cultured in complete RPMI-1640 medium were washed and seeded in glucose-free RPMI-1640 medium, as described above, and treated with or without 0.5 mM KCN (Merck, Germany) for seven hours with U-937 cells, or for five hours with PC-12 cells.

### 2. Testing of Elastase Inhibitor

200 μM elastase inhibitor III (MeOSuc-Ala-Ala-Pro-Val-CMK from Calbiochem) when added was administered 30 min before addition of the inducers. The inhibitor was dissolved in DMSO to a concentration of 100 mM. The final concentration of DMSO in the system was 0.2%, and was added to all treatments. In separate experiments, 200 μM of an elastase inhibitor (CE1037, manufactured by Cortech Inc.) was administered 30 min before addition of the inducers. The inhibitor was dissolved in PBS.

### 3. Cell Death Assay

### Trypan Blue Exclusion

At each time point, cell viability was determined by the trypan blue exclusion method (Daniel C P, Parreira A., et al. Leukemia Res. 11:191-196 (1987). Assays were performed in duplicate.

### Morphological Quantification of Apoptosis and Necrosis

Cells undergoing morphological changes associated with apoptotic or necrotic cell death were monitored as described by McGahon et al. [Methods Cell Biol, 46: p. 153-85 (1995)]. Briefly, 1 ml of the cells was collected and centrifuged. The pellet was resuspended in a 20-fold dilution of the dye mixture (composed of 100 μg/ml acridine orange and 100 μg/ml ethidium bromide in PBS), placed on a glass slide and viewed on an inverted fluorescence microscope. A minimum of 200 cells was scored for each sample.

### Preparation of Cell Lysates

4×10⁷ U-937 cells, treated or untreated with the various inducers, were collected after three hours of incubation, washed twice with ice-cold PBS and resuspended at 10⁸/ml in ice-cold lysing buffer (50 nM Tris-HCl pH 7.5, 0.1% NP-40, 1 mM DTT, 100 μM leupeptin and 100 μM TLCK). The cells were broken by the use of a polytron device (4 cycles of 7 seconds each) on ice, and the debris was pelleted by centrifugation in an ultracentrifuge at 120,000×g for 30 minutes at 4° C. The supernatant was used for further studies or stored at −70° C. The protein content of each sample was determined by the protein assay (BioRad).

### 5. Electrophoresis

Electrophoresis on a gelatin substrate gel was performed as previously described (Distefano J. F., Cotto C. A., et al. Cancer Invest. 6, 487-498, (1988)). Proteases were reversibly inactivated by addition of 100 μl aliquots of the cell lysates containing 200 μg protein to 50 μl of 0.625 M Tris-HCl buffer, pH 6.8, with 2.5% SDS, 10% sucrose and 0.03% phenol red. Samples were then electrophorated using 0.1% gelatin copolymerized in 11% polyacrylamide gel. After electrophoresis, the gels were subjected to three repeated immersions in 0.1 M Tris-HCl buffer, pH 7.0, containing 2.5% (V/V) Triton-x-100 in order to remove the SDS and reactivate the proteases. The gels were sliced and incubated overnight at 37° C. in 0.1 M glycine-NaOH buffer, pH 7.0, with or without 100 μM TPCK (chymotrypsin-like serine protease inhibitor) and 100 μM elastinal (elastase-like serine protease inhibitor). The bands of protease activity were developed with amido black staining.

### Results

### 1. Anti-Fas-Induced Apoptosis/Necrosis in U-937 Cells

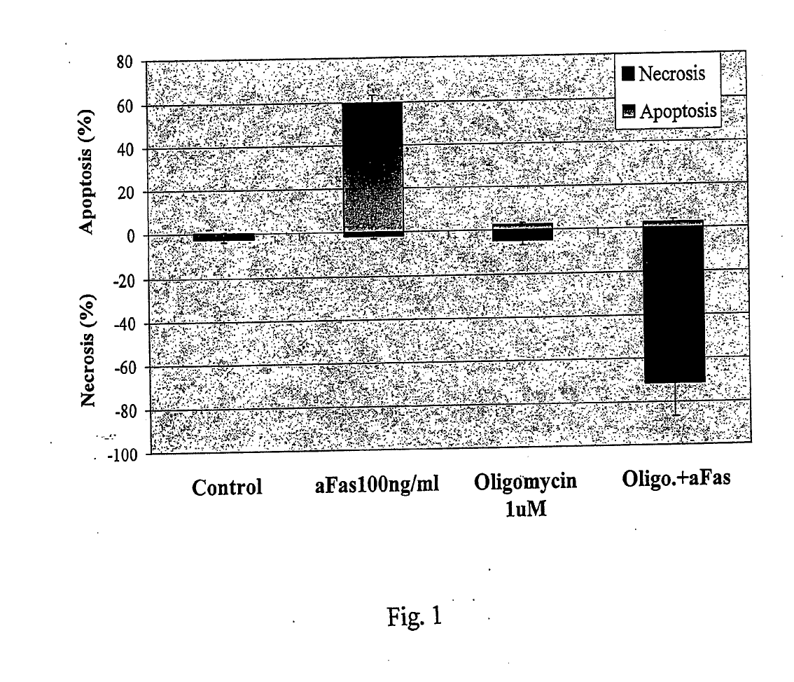

FIG. 1 indicates that treatment with anti-Fas induced about 60% apoptosis as compared to the control. Oligomycin is inactive by itself, however, addition of 100 ng/ml anti-Fas to oligomycin switched apoptotic cell death to necrotic cell death. Under these conditions, about 70% necrosis occurred and apoptosis returned to control level. Nuclear morphology was determined and analyzed by fluorescence microscope after double-staining with acridine orange and ethydium bromide.

### 2. Induction of Elastase-Like Activity During Necrotic Cell Death Induced by Anti-Fas in the Presence of Oligomycin

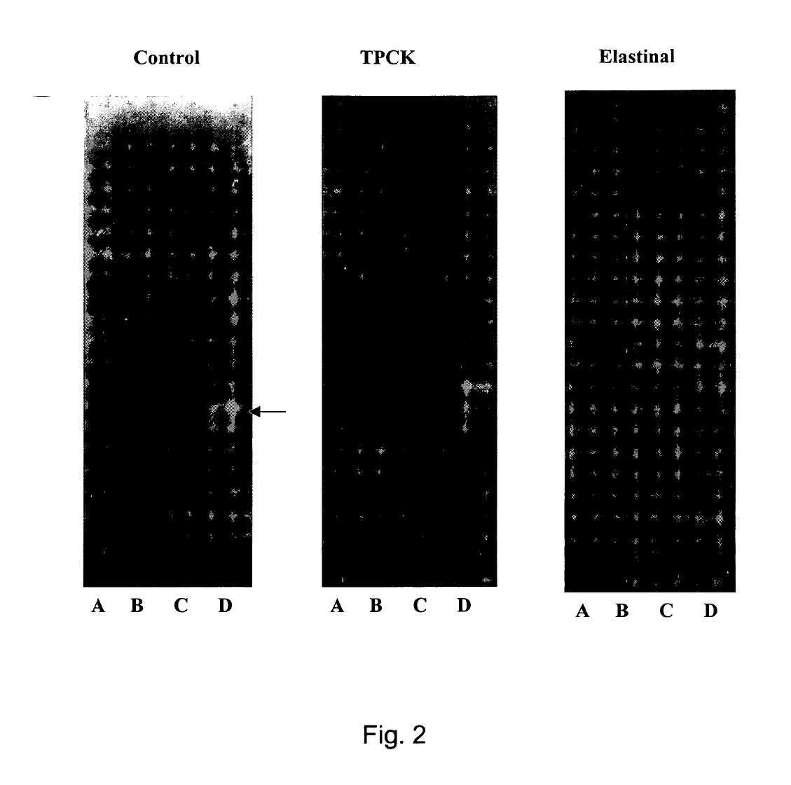

U-937 cells were maintained in glucose-free medium preincubated with or without 1 μM oligomycin for 45 min and treated with or without 100 ng/ml anti-Fas for three hours. Following this, cell lysates were prepared as described in “Experimental protocol” and applied to a gelatine substrate gel electrophoresis. The results, which are presented in FIG. 2 indicate that treatment with anti-Fas and oligomycin caused the appearance of a band of protease activity (line D), which was not found in the untreated control cells (lane A), anti-Fas-treated cells (lane B), or oligomycin-treated cells (lane C). This band disappeared in the presence of 100 μM elastinal (lane D), but not in the presence of 100 μM TPCK (lane D), indicating that treatment with anti-Fas and oligomycin induced an elastase-like activity, but not a chymotrypsin-like activity.

### 3. Induction of Elastase-Like Activity During Necrotic Cell Death Induced by KCN

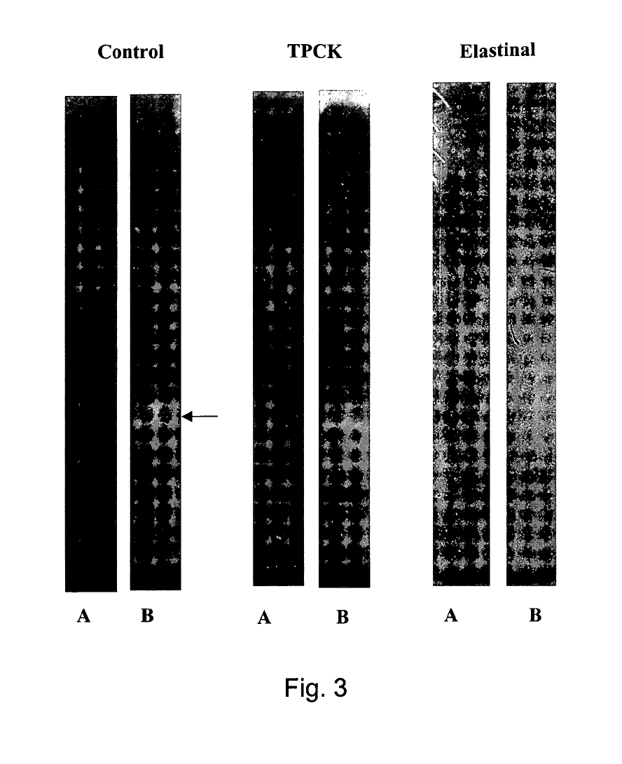

U-937 cells were treated with or without 0.5 mM KCN for three hours and then cell lysates were prepared as described in “Experimental protocol” and applied to a gelatine substrate gel electrophoresis. The results, which are presented in FIG. 3, show that treatment with KCN caused the appearance of a band of protease activity (lane B), which was not found in the untreated control cells (lane A). This band disappeared in the presence of 100 μM elastinal (lane B), but not in the presence of 100 μM TPCK (lane B), indicating that treatment with KCN induced an elastase-like activity, but not a chymotrypsin-like activity.

### 4. Effect of Elastase Inhibitor on Induction of Elastase-Like Activity During Necrotic Cell Death

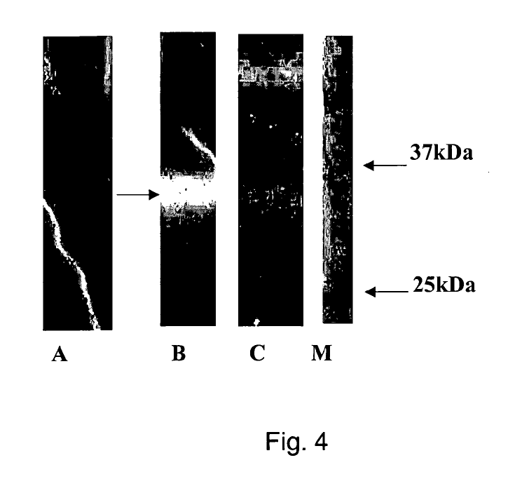

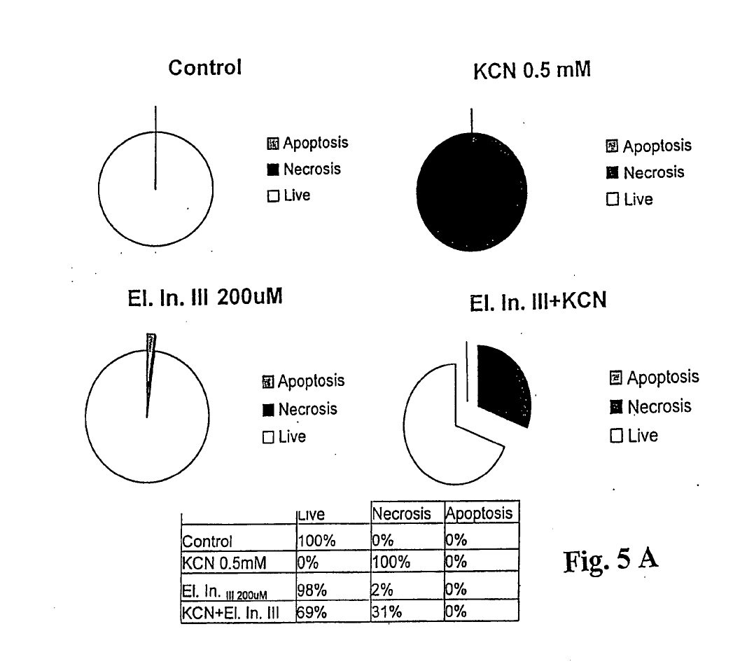

U-937 cells were treated with or without 5 mM KCN. 200 μM elastase inhibitor (Cortech) was added for three hours and then cell lysates were prepared as described in “Experimental protocol” and applied to a gelatine substrate gel electrophoresis. The results are presented in FIG. 4. It can be seen that treatment with KCN caused the appearance of a band of protease activity (lane B), which was not found in the untreated control cells (lane A). This band disappeared when KCN was administered in the presence of 200 μM elastase inhibitor (lane C).

### 5. Prevention of KCN-Induced Necrosis by Elastase Inhibitor III in PC-12 Cells

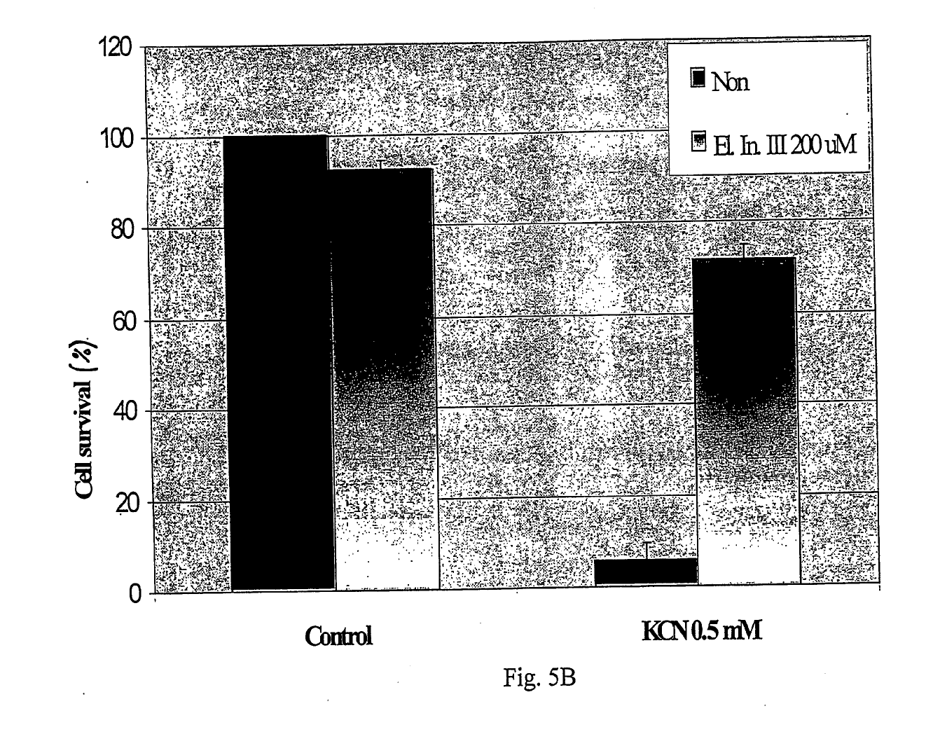

Exposure of PC-12 cells to 0.5 mM KCN induced massive necrotic cell death compared to the control. Addition of elastase inhibitor III which was inactive by itself significantly inhibited necrosis induced by KCN (FIG. 5, B). The protective effect of elastase inhibitor III is also seen when cell survival was determined under the same conditions by trypan blue exclusion (FIG. 5, A).

### 6. Inhibitory Effect of Elastase Inhibitor III on KCN-Induced Necrosis in U-937 Cells

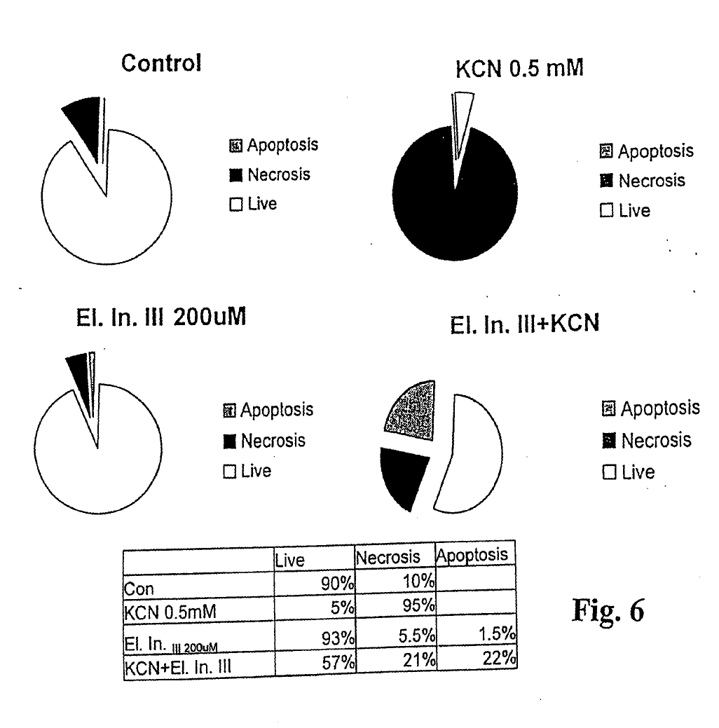

Treatment with KCN caused 95% necrosis as compared to 10% in the control. Addition of elastase inhibitor III with KCN markedly reduced necrotic cell death to 21%, and shifted 22% of the necrotic cell death to apoptotic cell death. 52% of the cells were protected from necrotic cell death by this inhibitor. Elastase inhibitor III did not cause any cell damage (FIG. 6).

### 7. Inhibitory Effect of Permeable Versus Non-Permeable Elastase Inhibitor on Anti-Fas-Induced Necrosis

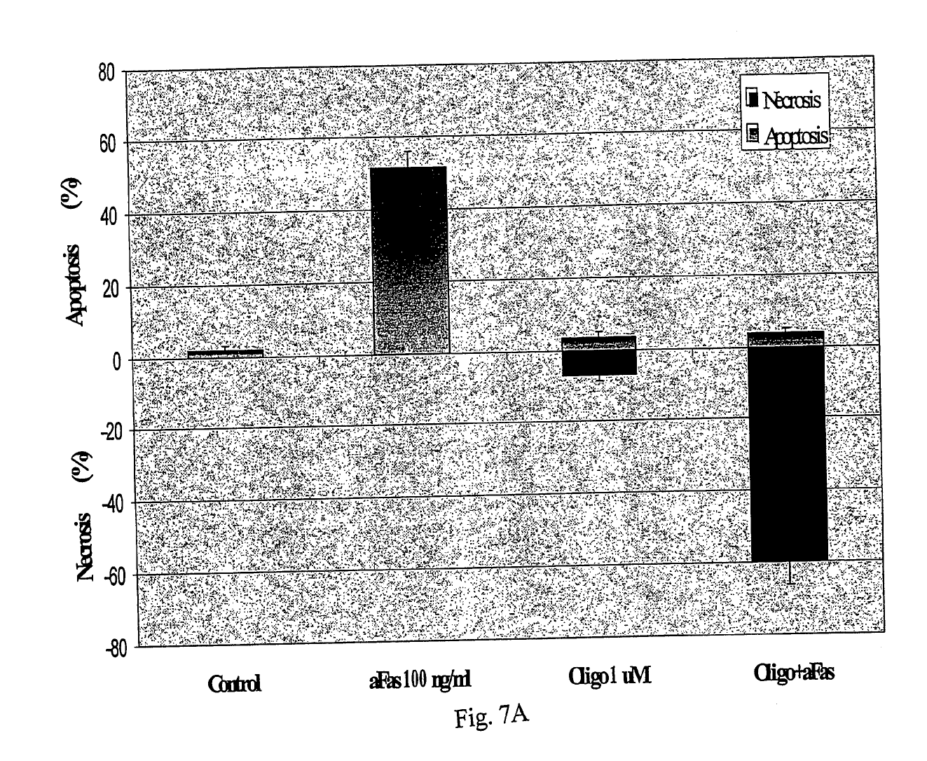

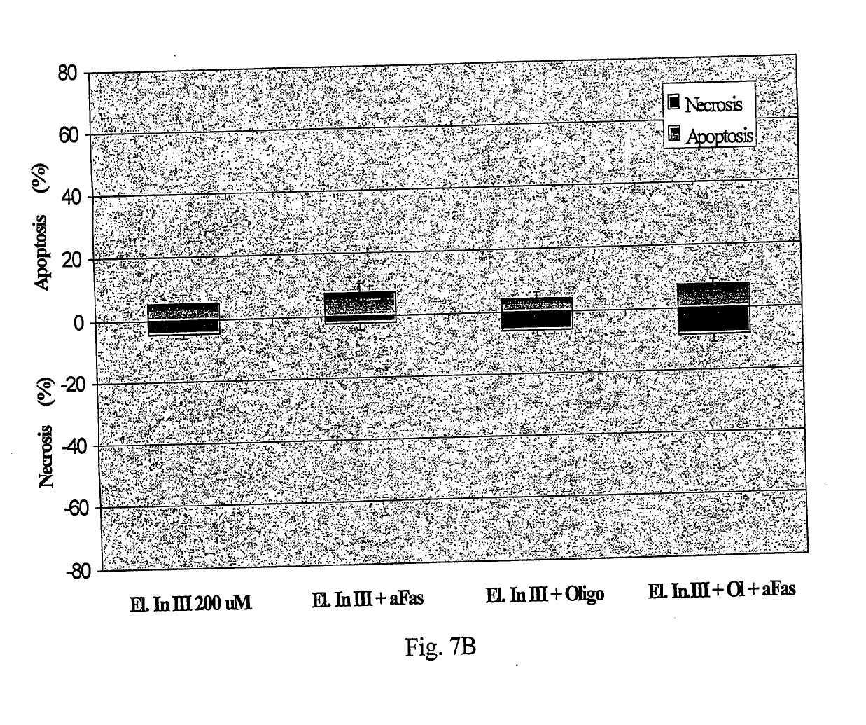

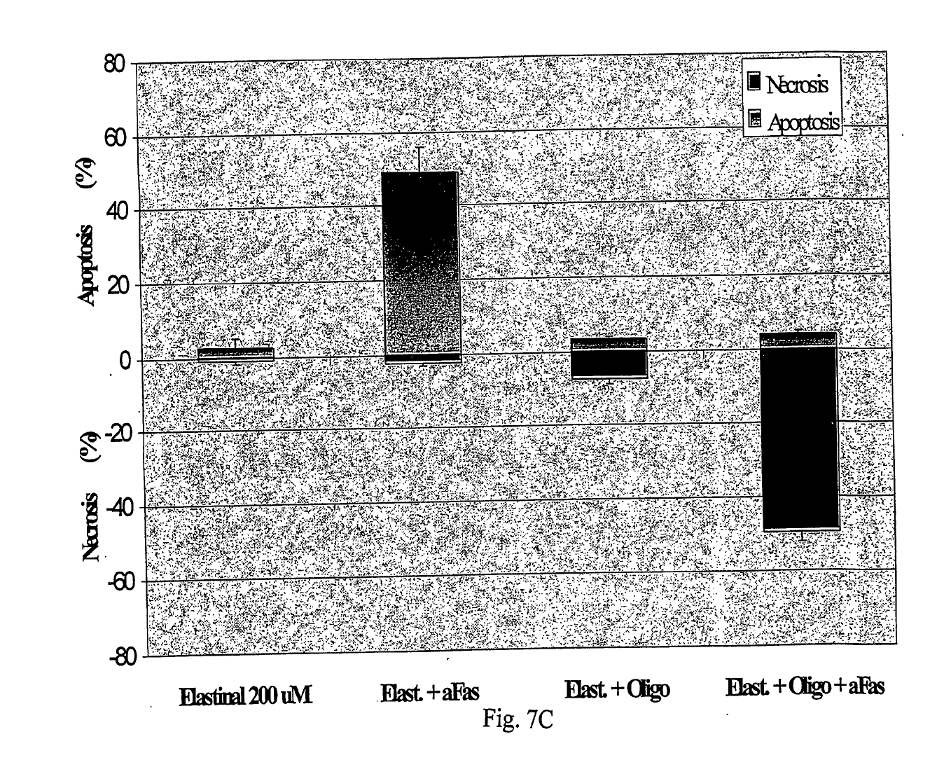

FIG. 7A shows anti-Fas-induced apoptosis/necrosis. Under these conditions cells were exposed to a permeable elastase inhibitor (Cortech Inc.). This exposure completely abrogated apoptotic as well as necrotic cell death (FIG. 7B). The non permeable elastase inhibitor-elastinal had no effect in this system (FIG. 7C).

### 8. STS-Induced Apoptosis/Necrosis in PC-12 Cells

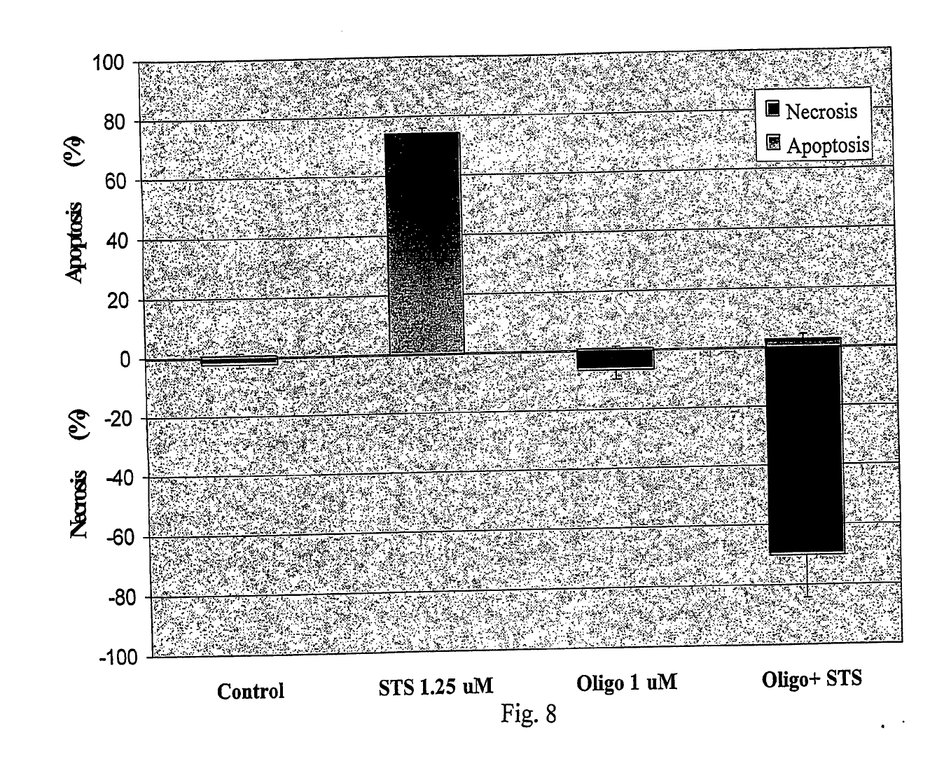

FIG. 8 indicates that treatment with 1.25 μM STS induced about 73% apoptosis as compared to the control. Oligomycin is inactive by itself, however, addition of STS to oligomycin switched apoptotic cell death to necrotic cell death. Under these conditions, about 70% necrosis occurred and apoptosis returned to control level. Nuclear morphology was determined and analyzed by fluorescence microscope after double-staining with acridine orange and ethidium bromide.

### 9. Inhibition of STS-Induced Apoptosis by Elastase Inhibitor in PC-12 Cells

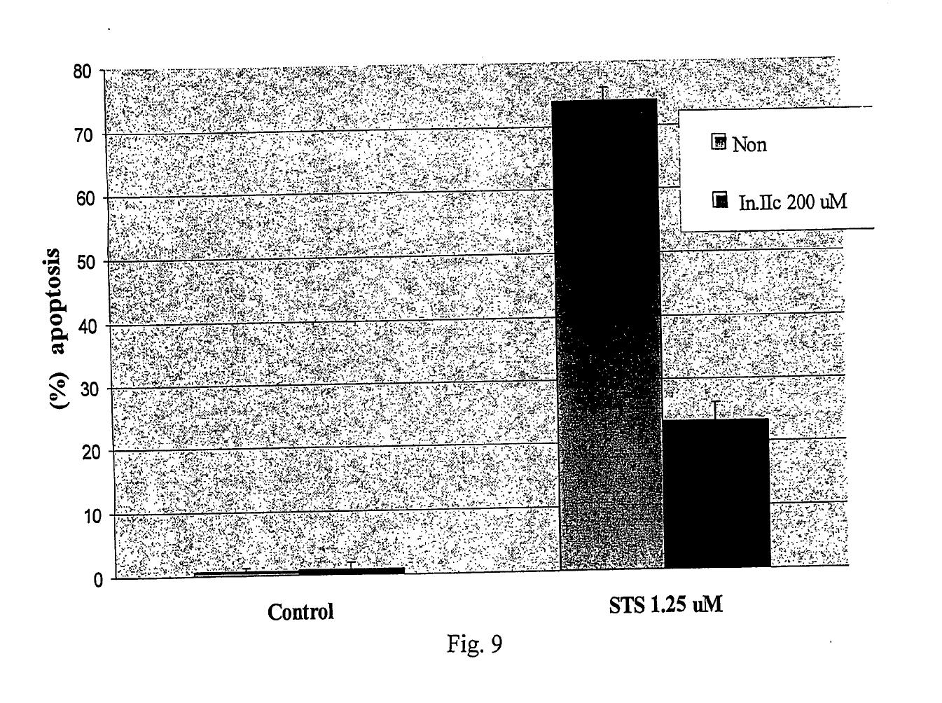

Exposure of PC-12 cells to 1.25 μM STS induced massive apoptotic cell death as compared to the control. Addition of 200 μM elastase inhibitor (Cortech, Inc.) which was inactive by itself significantly inhibited apoptosis induced by STS (FIG. 9).

### 10. Prevention of STS-Induced Necrosis by Elastase Inhibitor in PC-12 Cells

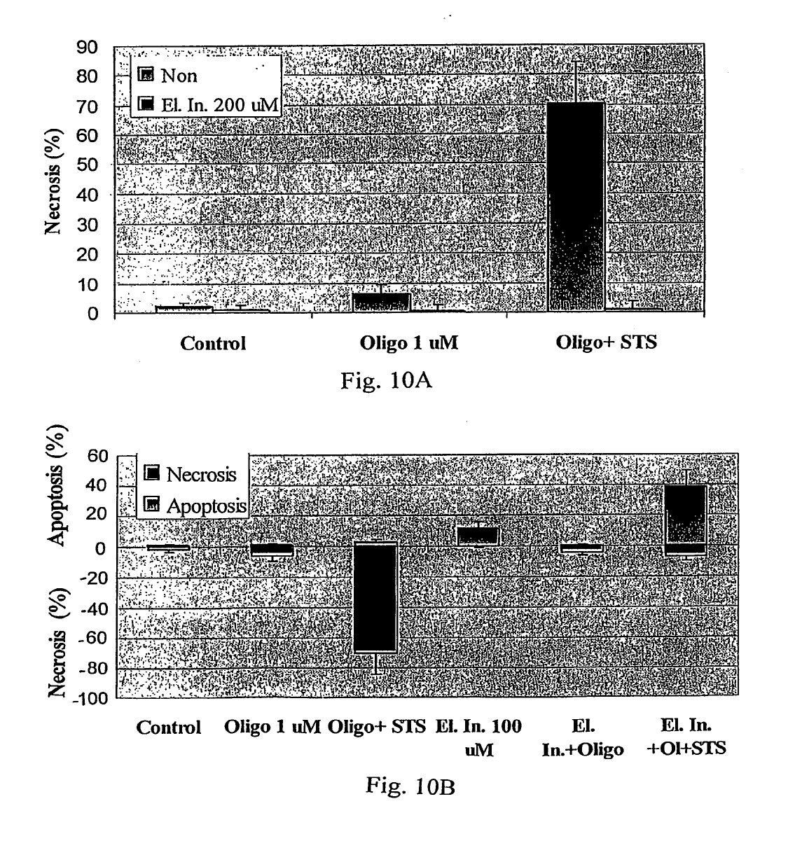

As seen in FIG. 10 A, 1.25 μM STS with 1 μM oligomycin induced about 70% necrosis. 200 μM elastase inhibitor was inactive by itself, but completely abrogated necrosis-induced by STS. Under the same conditions 100 μM elastase inhibitor markedly reduced necrotic cell death to 9%, and shifted 39% of the necrotic cell death to apoptotic cell death (FIG. 10B).

### 11. Inhibitory Effect of Elastase Inhibitor on KCN-Induced Necrosis in PC-12 Cells

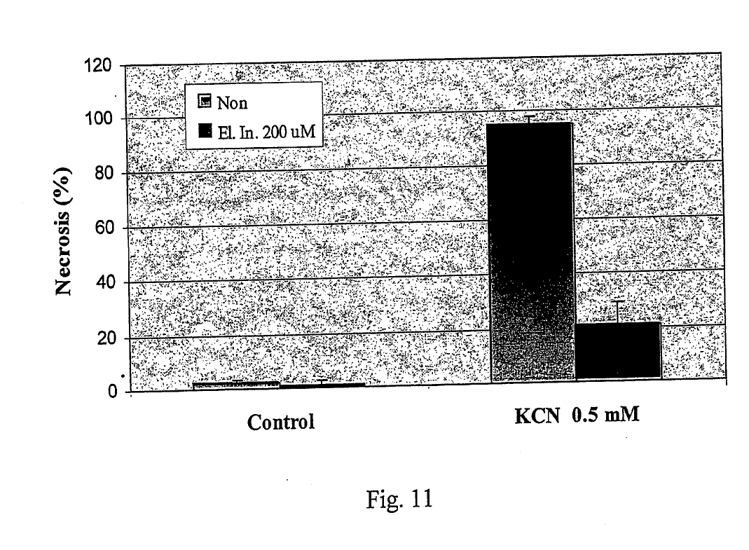

Exposure of PC-12 cells to 0.5 mM KCN induced massive necrotic cell death as compared to the control. Addition of 200 μM elastase inhibitor which was inactive by itself significantly inhibited necrosis induced by KCN (FIG. 11).

### 12. Effect of Elastase Inhibitor on STS-Induced Necrosis in U-937 Cells

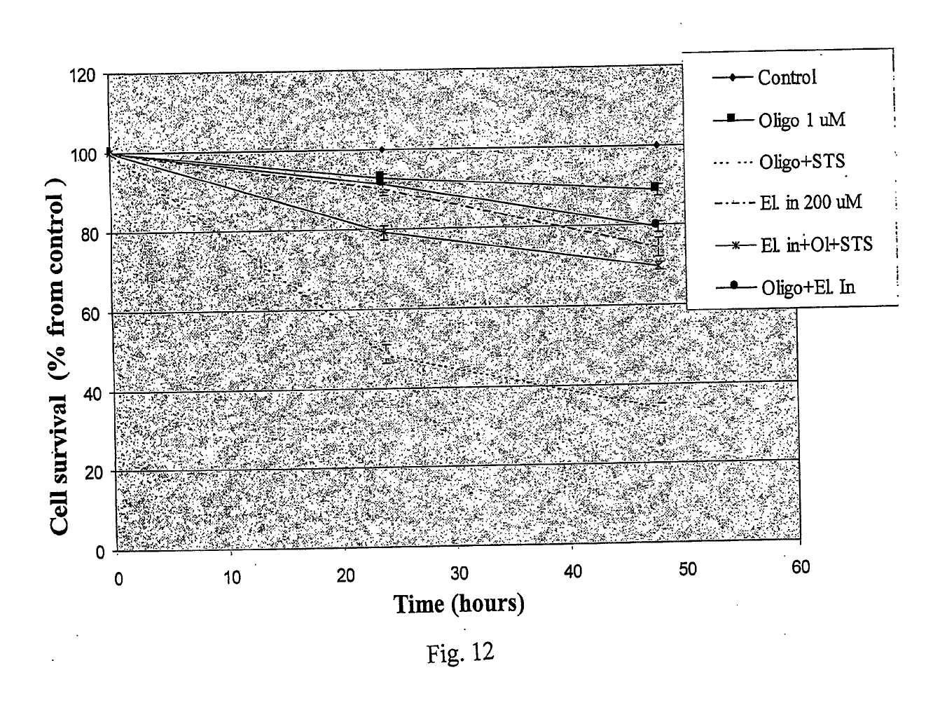

As seen in FIG. 12 treatment with STS in the presence of oligomycin markedly reduced cell survival as compared to control. Elastase inhibitor had a slight effect by itself, but it significantly inhibited cell killing induced by STS and oligomycin. The inhibitory effect was measured during prolong incubation of 48 hours. Cell viability was measured by trypan blue exclusion. Similar results were obtain for apoptosis (Data not shown).

1. A method of inhibiting or preventing necrosis of cells in a subject having leukemia or paracetamol poisoning, the method comprising administering to the subject a therapeutically effective amount of an elastase inhibitor that inhibits elastase activity in necrotic cells of the subject thereby inhibiting or preventing necrosis of cells.

2. (canceled)

3. The method of claim 1, wherein an amount of an elastase inhibitor is selected to cause conversion of cell necrosis to cell apoptosis.

4. The method of claim 3, further comprising administering to the subject an anti-apoptotic agent following or concomitantly with administering said elastase inhibitor.

5. The method of claim 4, wherein said anti-apoptotic agent is selected from the group consisting of—[R]—N-[2-heptyl]-methylpropargylamine (R-2HMP), vitamin E, vitamin D, caspase inhibitors and the hydrophilic bile salt ursodeoxycholic acid.

6. A method of inhibiting necrosis of cells in a subject having leukemia or paracetamol poisoning, the method comprising:
(a) administering to the subject an amount of an elastase inhibitor that causes conversion of cell necrosis to cell apoptosis, wherein said elastase inhibitor inhibits elastase activity in necrotic cells of the subject; and
(b) administering to the subject an anti-apoptotic agent, thereby inhibiting necrosis of cells in the subject.

7. The method of claim 6, wherein said anti-apoptotic agent is selected from the group consisting of—[R]—N-[2-heptyl]-methylpropargylamine (R-2HMP), vitamin E, vitamin D, caspase inhibitors and the hydrophilic bile salt ursodeoxycholic acid.

8. (canceled)

9. A method of inhibiting necrosis of cells in a subject having leukemia or paracetamol poisoning, the method comprising administering to the subject an amount of an elastase inhibitor that causes conversion of cell necrosis to cell apoptosis in necrotic cells of the subject, thereby inhibiting necrosis of cells.

10. (canceled)

11. A pharmaceutical composition comprising as active agents an elastase inhibitor and an anti-apoptotic agent and a pharmaceutically acceptable carrier.

12. The pharmaceutical composition of claim 11, wherein said anti-apoptotic agent is selected from the group consisting of—[R]—N-[2-heptyl]-methylpropargylamine (R-2HMP), vitamin E, vitamin D, caspase inhibitors and the hydrophilic bile salt ursodeoxycholic acid.

13. (canceled)

14. The method of claim 1, wherein said elastase inhibitor comprises elastase inhibitor III.

15. The method of claim 6, wherein said elastase inhibitor comprises elastase inhibitor III.

16. The method of claim 9, wherein said elastase inhibitor comprises elastase inhibitor III.


SEQUENCE LISTING (1 sequence)

SEQ ID NO: 1 - protein, 4 aa, Artificial sequence
  Elastase inhibitor III
AAPV